(19) United States
(12) Patent Application Publication
(10) Pub. No.: US 2004/0052728 A1
(43) Pub. Date: Mar. 18, 2004

(54) Diagnostic imaging

(76) Inventors: Morten Eriksen, Oslo (NO); Jonny Ostensen, Oslo (NO); Sigmund Frigstad, Trondheim (NO)

Correspondence Address: Amersham Health, 101 Carnegie Center, Princeton, NJ, 08540, US

(21) Appl. No.: 10/404,629

(22) Filed: Apr. 1, 2003

Related U.S. Application Data
(63) Continuation of application No. 09/672,994, filed on Sep. 29, 2000, now abandoned.
Application No. 09/672,994 is a continuation of application No. PCT/GB99/01002, filed on Mar. 31, 1999.
(60) Provisional application No. 60/082,727, filed on Apr. 23, 1998.
(60) Provisional application No. 60/115,001, filed on Jan. 6, 1999.

(30) Foreign Application Priority Data
Mar. 31, 1998 (GB) 9806910.7
Oct. 21, 1998 (GB) 9823070.9

(51) Int. Cl. A61K 51/00; A61K 49/04

(52) U.S. Cl. 424/1.11; 424/9.3; 424/9.4; 424/9.52

(57) ABSTRACT

A method of contrast agent-enhanced imaging involving induction of vasomodification, e.g. by physical or pharmacological means, in which pre- and post-vasomodification images in respect of free-flowing contrast or tracer agent in a substantially steady state distribution are recorded as part of a single imaging sequence and are compared to identify any local variations resulting from changes in vascular volume caused by the vasomodification. Imaging techniques which may be employed include ultrasound imaging, magnetic resonance imaging, X-ray imaging and nuclear tracer techniques such as scintigraphy.

This invention relates to diagnostic imaging, more particularly to use of diagnostic imaging in visualising tissue abnormalities. These include abnormalities in tissue perfusion, especially cardiac perfusion, for example such as may result from arterial stenoses.

In the field of ultrasound imaging it is well known that contrast agents comprising dispersions of gas microbubbles are particularly efficient backscatterers of ultrasound by virtue of the low density and ease of compressibility of the microbubbles. Such microbubble dispersions, if appropriately stabilised, may permit highly effective ultrasound visualisation of, for example, the vascular system and tissue microvasculature, often at advantageously low doses of the contrast agent.

Whilst existing ultrasound contrast agent imaging techniques may provide information as to whether particular organs or regions thereof are perfused or not, they in general do not have the sensitivity to detect abnormalities in tissue perfusion (which may be defined as blood flow per unit of tissue mass) caused by moderate arterial stenoses. Such information, which is valuable in assessing areas of potential infarction and whether a patient may benefit from preventative methods and/or treatment, is currently obtained using imaging techniques such as scintigraphy, positron emission tomography or single photon emission computed tomography, employing radioisotopic perfusion tracers.

It is well known in radionuclide cardiac imaging that patients may be subjected to physical or pharmacological stress in order to enhance the distinction between normally perfused myocardial tissue and any myocardial regions supplied by stenotic arteries. Thus, whereas such stress induces vasodilatation and increased blood flow in healthy myocardial tissue, blood flow in underperfused tissue supplied by a stenotic artery is substantially unchanged as a result of the capacity for arteriolar vasodilatation being already exhausted by inherent autoregulation seeking to increase the restricted blood flow. These differences may give rise to corresponding differences in image intensity as a result of perfusion differences.

It is also known to apply physical or pharmacological stress in ultrasound imaging of the heart (i.e. echocardiography) in order to modify cardiac perfusion. Thus, for example, Martin et al., Ann. Intern. Med. 116(3) (1992), pp. 190-196 report a stress echocardiography technique involving ultrasonic detection of left ventricular wall motion before and after administration of adenosine, dipyridamole or dobutamine; development of new or progressive wall motion abnormalities during such pharmacologically induced stress is said to be indicative of coronary disease.

A contrast agent-enhanced ultrasound imaging technique for detection of regional perfusion abnormalities during adenosine stress echocardiography is described by Kricsfeld et al. in J. Am. Coll. Cardiol. (Special Issue February 1995), p. 38A, Abstract 703-2. A contrast agent comprising perfluoropropane-enhanced sonicated dextrose albumin was intravenously administered to open-chested dogs either under resting conditions or during peak adenosine stress; the dogs either had no stenosis or had an angiographically significant stenosis in the proximal left circumflex coronary artery. It is reported that the ratios of peak myocardial videointensities obtained by ultrasonic imaging of the left circumflex perfusion beds in dogs subject to adenosine stress compared to resting dogs was in the range 1.8-2.1:1 for stenoses with diameter not exceeding 50% and in the range 0.8-1.4:1 for stenoses with diameter in excess of 70%. It will be appreciated that whilst this technique may permit some generalised identification of a region of abnormal perfusion, the fact that separate images are recorded in respect of stressed and resting animals inevitably means that detailed images in respect of perfusion abnormalities cannot be obtained.

More sophisticated methods for ultrasound perfusion imaging using a vasodilator drug are described in WO-A-9817324 and our copending and currently unpublished International Patent Application No. PCT/GB98/03155. These rely on use of ultrasound contrast agents capable of accumulation in tissue microvasculature, for example as a result of controlled temporary microbubble growth in vivo. Such agents will accumulate in tissue in concentrations related to the regional rate of tissue perfusion, so that ultrasound imaging modalities such as conventional or harmonic B-mode imaging, in which the display is derived from return signal intensities, will provide images which may be interpreted as perfusion maps, since the displayed signal intensity will be a function of local perfusion. Coadministration of a vasodilator drug with such accumulating ultrasound contrast agents substantially enhances contrast agent uptake in healthy tissue, for example in the myocardium, but not in hypoperfused tissue supplied by a stenotic artery; the ratio between return signal intensities from normal tissue and hypoperfused tissue may therefore be significantly increased.

It will be appreciated that ultrasound contrast agents which are not capable to any significant extent of accumulation in tissue microvasculature, hereinafter referred to as “free-flowing contrast agents”, exhibit fundamentally different behaviour, since the regional concentration of such free-flowing agents and the return signal intensity therefrom will depend on actual blood content within imaged tissue rather than the local rate of perfusion.

The present invention is based on the surprising finding that valuable and detailed information regarding perfusion and other tissue abnormalities maybe obtained using a variety of imaging techniques employing free-flowing contrast or tracer agents in conjunction with a range of vasodilatation- or vasoconstrictor-inducing or other vasoregulation-modifying techniques., which for brevity are hereinafter referred to as “vasomodification-inducing techniques”. The method of the invention relies on the use of such free-flowing agents to determine relative changes in vascular volume cause by such vasomodification-inducing techniques. The determination of relative changes in vascular volume induced by factors such as physical or pharmacological stress has not hitherto been used as a marker for disease, and represents a key feature of the present invention.

In contradistinction to existing imaging methods such as contrast agent-enhanced stress echocardiography, which generally involve images obtained using contrast agent administered during or after induction of vasodilatation, the method of the present invention induces vasomodification after contrast or tracer agent-enhanced imaging has been begun. Thus, if the contrast or tracer agent is substantially free-flowing in vivo and remains or is maintained in a substantially steady state distribution in the blood stream during the course of the imaging procedure, a comparison of regional signal intensity in images recorded before and after the onset of vasomodification will permit detection of changes in vascular volume caused by the vasomodification. Healthy tissue will be characterised by a significant change in signal intensity, whereas the signal intensity from hypoperfused tissue will remain relatively unchanged because of the autoregulation-induced inability of such tissue to undergo significant vasomodification. Because the pre- and post-vasomodification images are recorded as part of a single overall sequence and are closely spaced temporally, it is possible to ensure their close alignment in any subsequent image processing procedures, so that results with a high degree of robustness may be obtained. Furthermore, since factors such as blood concentration of contrast or tracer agent, tissue geometry and, where appropriate, signal attenuation, all of which may influence signal intensity from tissue, may be maintained substantially constant during the overall imaging procedure, the observed changes in signal intensity may be used to provide a direct quantitative indication of changes in vascular volume.

According to one aspect thereof the present invention provides a method for detection of abnormalities in vasculated tissue within a human or non-human animal subject which comprises (A) injecting a substantially free-flowing contrast or tracer agent into the vascular system of said subject so as to generate a substantially steady state distribution of said agent in the blood stream of said subject during the steps of:

(i) generating one or more first images in respect of vasculated tissue in a target area;

(ii) inducing vasomodification within said target tissue; and

(iii) generating one or more second images in respect of said vasomodified target tissue;

and (B) comparing said first and second images to identify any local variations in the change in signal intensity resulting from vascular volume changes induced by said vasomodification.

The invention further embraces the use of a free-flowing contrast or tracer agent and a vasomodification-inducing substance or means in the above-defined method and in the manufacture of a combined diagnostic formulation or regimen for use in the above-defined method.

Imaging techniques which may be used to visualise vascular volume changes in accordance with the invention include ultrasound imaging, magnetic resonance imaging, X-ray imaging and nuclear tracer techniques such as scintigraphy. Organs which may be studied include the liver, kidneys, brain and heart.

Free-flowing ultrasound contrast agents which may be used in ultrasound imaging in accordance with the invention include gas-containing and gas-generating formulations which give rise to echogenic gas microbubbles in the blood stream upon intravenous injection.

Gases which may be used include any biocompatible substances, including mixtures, which are at least partially, e.g. substantially or completely, in gaseous or vapour form at the normal human body temperature of 37EC. Representative gases thus include air; nitrogen; oxygen; carbon dioxide; hydrogen; inert gases such as helium, argon, xenon or krypton; sulphur fluorides such as sulphur hexafluoride, disulphur decafluoride or trifluoromethylsulphur pentafluoride; selenium hexafluoride; optionally halogenated silanes such as methylsilane or dimethylsilane; low molecular weight hydrocarbons (e.g. containing up to 7 carbon atoms), for example alkanes such as methane, ethane, a propane, a butane or a pentane, cycloalkanes such as cyclopropane, cyclobutane or cyclopentane, alkenes such as ethylene, propene, propadiene or a butene, and alkynes such as acetylene or propyne; ethers such as dimethyl ether; ketones; esters; halogenated low molecular weight hydrocarbons (e.g. containing up to 7 carbon atoms); and mixtures of any of the foregoing. Advantageously at least some of the halogen atoms in halogenated gases are fluorine atoms; thus biocompatible halogenated hydrocarbon gases may, for example, be selected from bromochlorodifluoromethane, chlorodifluoromethane, dichlorodifluoromethane, bromotrifluoromethane, chlorotrifluoromethane, chloropentafluoroethane, dichlorotetrafluoroethane, chlorotrifluoroethylene, fluoroethylene, ethylfluoride, 1,1-difluoroethane and perfluorocarbons. Representative perfluorocarbons include perfluoroalkanes such as perfluoromethane, perfluoroethane, perfluoropropanes, perfluorobutanes (e.g. perfluoro-n-butane, optionally in admixture with other isomers such as perfluoro-iso-butane), perfluoropentanes, perfluorohexanes or perfluoroheptanes; perfluoroalkenes such as perfluoropropene, perfluorobutenes (e.g. perfluorobut-2-ene), perfluorobutadiene, perfluoropentenes (e.g. perfluoropent-1-ene) or perfluoro-4-methylpent-2-ene; perfluoroalkynes such as perfluorobut-2-yne; and perfluorocycloalkanes such as-perfluorocyclobutane, perfluoromethylcyclobutane, perfluorodimethylcyclobutanes, perfluorotrimethylcyclobutanes, perfluorocyclopentane, perfluoromethylcyclopentane, perfluorodimethylcyclopentanes, perfluorocyclohexane, perfluoromethylcyclohexane or perfluorocycloheptane. Other halogenated gases include methyl chloride, fluorinated (e.g. perfluorinated) ketones such as perfluoroacetone and fluorinated (e.g. perfluorinated) ethers such as perfluorodiethyl ether. The use of perfluorinated gases, for example sulphur hexafluoride and perfluorocarbons such as perfluoropropane, perfluorobutanes, perfluoropentanes and perfluorohexanes, may be particularly advantageous in view of the recognised high stability in the blood stream of microbubbles containing such gases. Other gases with physicochemical characteristics which cause them to form highly stable microbubbles in the blood stream may likewise be useful.

Representative examples of contrast agent formulations include microbubbles of gas stabilised (e.g. at least partially encapsulated) by a coalescence-resistant surface membrane (for example gelatin, e.g. as described in WO-A-8002365), a filmogenic protein (for example an albumin such as human serum albumin, e.g. as described in U.S. Pat. No. 4,718,433, U.S. Pat. No. 4,774,958, U.S. Pat. No. 4,844,882, EP-A-0359246, WO-A-9112823, WO-A-9205806, WO-A-9217213, WO-A-9406477, WO-A-9501187 or WO-A-9638180), a polymer material (for example a synthetic biodegradable polymer as described in EP-A-0398935, an elastic interfacial synthetic polymer membrane as described in EP-A-0458745, a microparticulate biodegradable polyaldehyde as described in EP-A-0441468, a microparticulate N-dicarboxylic acid derivative of a polyamino acid polycyclic imide as described in EP-A-0458079, or a biodegradable polymer as described in WO-A-9317718 or WO-A-9607434), a non-polymeric and non-polymerisable wall-forming material (for example as described in WO-A-9521631), or a surfactant (for example a polyoxyethylene-polyoxypropylene block copolymer surfactant such as a Pluronic, a polymer surfactant as described in WO-A-9506518, or a film-forming surfactant such as a phospholipid, e.g. as described in WO-A9211873, WO-A-9217212, WO-A-9222247, WO-A-9409829, WO-A9428780, WO-A-9563835 or WO-A-9729783). Contrast agent formulations comprising free microbubbles of selected gases, e.g. as described in WO-A-9305819, or comprising a liquid-in-liquid emulsion in which the boiling point of the dispersed phase is below the body temperature of the subject to be imaged, e.g. as described in WO-A9416739, may also be used.

Other useful gas-containing contrast agent formulations include gas-containing solid systems, for example microparticles (especially aggregates of microparticles) having gas contained therewithin or otherwise associated therewith (for example being adsorbed on the surface thereof and/or contained within voids, cavities or pores therein, e.g. as described in EP-A-0122624, EP-A-0123235, EP-A-0365467, WO-A-9221382, WO-A-9300930, WO-A-9313802, WO-A-9313808 or WO-A-9313809). It will be appreciated that the echogenicity of such microparticulate contrast agents may derive directly from the contained/associated gas and/or from gas (e.g. microbubbles) liberated from the solid material (e.g. upon dissolution of the microparticulate structure).

The disclosures of all of the above-described documents relating to gas-containing contrast agent formulations are incorporated herein by reference.

Gas microbubbles and other gas-containing materials such as microparticles preferably have an initial average size not exceeding 10 μm (e.g. of 7 μm or less) in order to permit their free passage through the pulmonary system following administration, e.g. by intravenous injection. However, larger microbubbles may be employed where, for example, these contain a mixture of one or more relatively blood-soluble or otherwise diffusible gases such as air, oxygen, nitrogen or carbon dioxide with one or more substantially insoluble and non-diffusible gases such as perfluorocarbons. Outward diffusion of the soluble/diffusible gas content following administration will cause such microbubbles rapidly to shrink to a size which will be determined by the amount of insoluble/non-diffusible gas present and which may be selected to permit passage of the resulting microbubbles through the lung capillaries of the pulmonary system.

Where phospholipid-containing contrast agent formulations are employed in accordance with the invention, e.g. in the form of phospholipid-stabilised gas microbubbles, representative examples of useful phospholipids include lecithins (i.e. phosphatidylcholines), for example natural lecithins such as egg yolk lecithin or soya bean lecithin, semisynthetic (e.g. partially or fully hydrogenated) lecithins and synthetic lecithins such as dimyristoylphosphatidylcholine, dipalmitoylphosphatidylcholine or distearoylphosphatidylcholine; phosphatidic acids; phosphatidylethanolamines; phosphatidylserines; phosphatidylglycerols; phosphatidylinositols; cardiolipins; sphingomyelins; fluorinated analogues of any of the foregoing; mixtures of any of the foregoing and mixtures with other lipids such as cholesterol. The use of phospholipids predominantly (e.g. at least 75%) comprising molecules individually bearing net overall charge, e.g. negative charge, for example as in naturally occurring (e.g. soya bean or egg yolk derived), semisynthetic (e.g. partially or fully hydrogenated) and synthetic phosphatidylserines, phosphatidylglycerols, phosphatidylinositols, phosphatidic acids and/or cardiolipins, for example as described in WO-A-9729783, may be particularly advantageous.

Representative examples of materials useful in gascontaining contrast agent microparticles include carbohydrates (for example hexoses such as glucose, fructose or galactose; disaccharides such as sucrose, lactose or maltose; pentoses such as arabinose, xylose or ribose; α-, β- and γ-cyclodextrins; polysaccharides such as starch, hydroxyethyl starch, amylose, amylopectin, glycogen, inulin, pulullan, dextran, carboxymethyl dextran, dextran phosphate, ketodextran, aminoethyldextran, alginates, chitin, chitosan, hyaluronic acid or heparin; and sugar alcohols, including alditols such as mannitol or sorbitol), inorganic salts (e.g. sodium chloride), organic salts (e.g. sodium citrate, sodium acetate or sodium tartrate), X-ray contrast agents (e.g. any of the commercially available carboxylic acid and non-ionic amide contrast agents typically containing at least one 2,4,6-triiodophenyl group having substituents such as carboxyl, carbamoyl, N-alkylcarbamoyl, N-hydroxyalkylcarbamoyl, acylamino, N-alkylacylamino or acylaminomethyl at the 3- and/or 5-positions, as in metrizoic acid, diatrizoic acid, iothalamic acid, ioxaglic acid, iohexol, iopentol, iopamidol, iodixanol, iopromide, metrizamide, iodipamide, meglumine iodipamide, meglumine acetrizoate and meglumine diatrizoate), polypeptides and proteins (e.g. gelatin or albumin such as human serum albumin), and mixtures of any of the foregoing.

The nature of the gas and/or of any stabilising material is preferably selected so that the microbubbles are sufficiently stable to recirculate in the blood stream, for example for at least 30 seconds, preferably for at least one or two minutes, and thereby generate a substantially steady state distribution in the blood pool. In this way a steady state contrast effect, for example showing no apparent change in contrast intensity over a period of about 10 seconds, may be achieved in the equilibrium phase following administration of an appropriate bolus of contrast agent. A substantially steady state distribution may alternatively be obtained through continuous infusion of contrast agent, in which case the stability requirements for the contrast agent may be less critical.

Free-flowing magnetic resonance contrast agents which may be used in magnetic resonance imaging in accordance with the invention include substances containing non-zero nuclear spin isotopes such as ¹⁹F or having unpaired electron spins and hence paramagnetic, superparamagnetic, ferrimagnetic or ferromagnetic properties. These include magnetic iron oxide particles and chelated paramagnetic ions such as Gd, Dy, Fe and Mn, especially when chelated by macrocyclic chelant groups (eg. tetraazacyclododecane chelants such as DOTA, DO3A, HP-D03A and analogues thereof) or by linker chelant groups such as DTPA, DTPA-BMA, EDTA, DPDP, etc.

Free-flowing X-ray contrast agents which may be used in X-ray imaging in accordance with the invention include substances containing a heavy atom, e.g. of atomic weight 38 or above, for example chelated heavy metal cluster ions (e.g. tungsten or molybdenum polyoxyanions or their sulphur or mixed oxygen/sulphur analogues), covalently bonded non-metal atoms which either have high atomic number (e.g. such as iodine) or are radioactive, (e.g. ¹²³I or ¹³¹I atoms), or iodinated compound-containing vesicles.

Free-flowing tracer agents which may be used in nuclear tracer techniques in accordance with the invention will typically incorporate a metal radionuclide such as ⁹⁰Y, ^{99m}Tc, ¹¹¹In, ⁴⁷Sc, ³⁴Ga, ⁵¹Cr, ¹¹⁷Sn, ⁶⁷Cu, ¹⁶⁷Tm, ⁹⁷Ru, ¹⁸⁸Re, ¹⁷⁷Lu, ¹⁹⁹Au, ²⁰³Pb or ¹⁴¹Ce.

Representative examples of contrast agents which may be useful in the above imaging techniques are listed as possible reporters in WO-A-9818496 and WO-A-9818497, the contents of which are incorporated herein by reference. The use of microparticulate and/or high molecular weight contrast agents which are substantially retained within the vascular system is generally preferred.

Vasomodification may be induced in the target tissue by any suitable pharmacological or physical method, for example by administration of an appropriate vasoactive substance or by application of localised heating or cooling; the use of endogenous vasoactive substances may be advantageous. In general, vasoactive substances may be administered by any appropriate route, for example intravenously, intra-arterially, interstitially, topically or by selective catheterisation or iontophoresis. Use of substances or methods which lead to rapid onset of vasomodification is preferred, since this will minimise the overall time needed to maintain substantially steady state distribution of contrast or tracer agent and to obtain the pre- and post-vasomodification images; consistency between the two sets of images may thereby be enhanced. It will be appreciated that it is not necessary in operating the method of the invention for the effects of the vasoactive substance or method to be confined only to the target tissue.

Vasoactive substances which may be employed include vasodilators, vasoconstrictors, hormones, local signal substances and receptor blockers. They may, for example, act directly on the vascular system or may indirectly induce changes in perfusion and vascular volume, e.g. by increasing metabolism.

Vasodilators are a preferred category of vasoactive substances useful in accordance with the invention. Administration of a vasodilator drug will result in a significant increase in signal intensity in images from healthy tissue, whereas the signal intensity from hypoperfused tissue will remain relatively unchanged or may even decrease as a result of “steal” phenomena.

Representative vasodilator drugs useful in accordance with the invention include endogenous/metabolic vasodilators such as lactic acid, adenosine triphosphate, adenosine diphosphate, adenosine monophosphate, adenosine, nitric oxide and agents causing hypercapnia, hypoxia/hypoxemia or hyperemia; phosphodiesterase inhibitors such as dipyridamole and sildenafil; sympathetic activity inhibitors such as clonidine and methyldopa; smooth muscle relaxants such as papaverine, hydralazine, dihydralazine and nitroprusside; beta receptor agonists such as dopamine, dobutamine, arbutamine, albuterol, salmeterol and isoproterenol; alpha receptor antagonists such as doxazosin, terazosin and prazosin; organic nitrates, such as glyceryl trinitrate, isosorbide dinitrate and isosorbide mononitrate; angiotensin converting enzyme (ACE) inhibitors such as benazepril, captopril, enalapril, fosinopril, lisinopril, quinapril and ramipril; angiotensin II antagonists (or AT1 receptor antagonists) such as valsartane, losartan and candesartan; calcium channel blockers such as amlodipine, nicardipine, nimodipine, felodipine, isradipine, diltiazem, verapamil and nifedipine; prostaglandins such as alprostadil; and endotheliumdependent vasodilators.

Use of adenosine is particularly preferred since it is an endogenous substance and has a rapid but short-lived vasodilatating effect. This latter property is confirmed by the fact that it has a blood pool half-life of only a few seconds; possible discomfort to patients during vasodilatation is therefore minimised. Vasodilatation induced by adenosine will be most intense in the heart since the drug will tend to reach more distal tissues in less than pharmacologically active concentrations; it is therefore the vasodilator drug of choice in cardiographic applications of the method of the invention.

In addition to arterial stenoses, other tissue/perfusion abnormalities which affect local vasoregulation may be detectable in accordance with the invention. Thus, for example, vessels within malignant lesions are known to be poorly differentiated and may therefore exhibit impaired response to vasoconstrictor drugs compared to normal tissue; a similar lack of vasoconstrictory response may occur in severely inflamed tissue. Observation of the response to a vasoconstrictor stimulus in terms of changes in signal intensity during an imaging procedure may therefore give useful diagnostic information. Representative examples of vasoconstrictor drugs which may be useful in such embodiments include isoprenaline, epinephrine, norepinephrine, dopamine, metaraminol, prenalterol, ergotamine, dihydroergotamime, methysergide and inhibitors of nitric oxide production, such as analogues of L-arginine; such drugs may, for example, be administered either locally or systemically.

For some purposes it may be advantageous to administer two or more vasoactive substances, either together or in sequence. When two vasoactive substances are applied, both may be vasodilators, both may be vasoconstrictors, or one may be a vasodilator and the other may be a vasoconstrictor. When two vasoactive substances belonging to the same class are used (both vasodilators or both vasoconstrictors), they should differ in at least one property, such as tissue specificity or mechanism of action, so that local differences in signal intensity may be determined during a single examination. When administered separately, a vasoconstrictor may first be administered, followed by a vasodilator, or the reverse order may be used.

In cardiographic procedures it may be advantageous to use an ECG-gated intermittent imaging procedure to record the pre- and post-vasomodification images. Image quality may also be improved if the subject holds his or her breath during the imaging procedure; this should not create compliance problems where rapid-acting short-lived vasodilator drugs such as adenosine are employed.

Vasodilatation in healthy tissue may typically increase the vascular volume fraction from a baseline of about 8% to a peak value of about 15%, thereby leading to an approximately two-fold increase in signal intensity, i.e. 3dB in the case of ultrasound imaging techniques. Larger increases (e.g. up to four- or fivefold) may be obtained using potent vasodilator drugs such as adenosine. It is therefore possible to make a direct visual comparison of signal intensities in preand post-vasodilatation images such as ultrasound images in order to distinguish between healthy and hypoperfused tissue. Alternatively or additionally the two images or sets of images may be compared by division or subtraction of appropriate signal intensity parameters; it will be appreciated that subtraction of logarithmic values such as decibel changes in ultrasound imaging will effectively correspond to division. By way of example, the two images or sets of images may be subjected to appropriate time domain image processing, techniques, for example conventional techniques such as image filtering and subtraction, if desired with additional automated geometric fitting to minimise any effect of misalignment between the images (although the fact that the images are recorded as part of a single overall sequence and are closely spaced temporally will inherently tend to keep such misalignment to a minimum).

In the case of ultrasound imaging, image subtraction-derived results may, for example, be presented as a smoothed integrated backscatter difference image, e.g. with contour lines corresponding to 3 dB changes in signal intensity or with pseudo-colouring for each 3 dB range of change in signal intensity.

The sensitivity of the method of the invention is such that it may permit detection of moderate as well as severe arterial stenoses in any tissue area of the body, particularly in the heart.

In ultrasound imaging, the use of ultrasound irradiation at intensities known to cause destruction of the contrast agent may further improve the diagnostic potential of the method; under such conditions changes in returned echo intensities from the contrast agent may show an increased dependency on perfusion, with an increased relative change during vasomodification.

Representative ultrasound imaging techniques which may be useful in accordance with the invention include fundamental B-mode imaging; harmonic B-mode imaging including reception of sub-harmonics and the second and higher harmonics; tissue Doppler imaging, optionally including selective reception of fundamental, harmonic or sub-harmonic echo frequencies; colour Doppler imaging, optionally including selective reception of fundamental, harmonic or sub-harmonic echo frequencies; power Doppler imaging, optionally including selective reception of fundamental, harmonic or sub-harmonic echo frequencies; power or colour Doppler imaging utilising loss of correlation or apparent Doppler shifts caused by changes in the acoustical properties of contrast agent microbubbles such as may be caused by spontaneous or ultrasound-induced destruction, fragmentation, growth or coalescense; pulse inversion imaging, optionally including selective reception of fundamental, harmonic or sub-harmonic echo frequencies, and also including techniques wherein the number of pulses emitted in each direction exceeds two; pulse inversion imaging utilising loss of correlation caused by changes in the acoustical properties of contrast agent microbubbles such as may be caused by spontaneous or ultrasound-induced destruction, fragmentation, growth or coalescense; pulse pre-distortion imaging, e.g. as described in 1997 IEEE Ultrasonics Symposium, pp. 1567-1570; and ultrasound imaging techniques based on comparison of echoes obtained with different emission output amplitudes or waveform shapes in order to detect non-linear effects caused by the presence of gas bubbles.

The following non-limitative examples serve to illustrate the invention.

Preparation 1—Hydrogenated phosphatidylserine-encapsulated Perfluorobutane Microbubbles

Hydrogenated phosphatidylserine (5 mg/ml in a 1% w/w solution of propylene glycol in purified water) and perfluorobutane gas were homogenised in-line at 7800 rpm and ca. 40Ec to yield a creamy-white microbubble dispersion. The dispersion was fractionated to substantially remove undersized microbubbles (2 μm) and the volume of the dispersion was adjusted to the desired microbubble concentration. Sucrose was then added to a concentration of 92 mg/ml. 2 ml portions of the resulting dispersion were filled into 10 ml flat-bottomed vials specially designed for lyophilisation, and the contents were lyophilised to give a white porous cake. The lyophilisation chamber was then filled with perfluorobutane and the vials were sealed. Prior to use, water was added to a vial and the contents were gently hand-shaken for several seconds to give a perfluorobutane microbubble dispersion containing 10 μl gas/ml.

Preparation 2—Perfluoropropane-Containing Human Serum Albumin Microspheres

A contrast agent formulation comprising perfluoropropane-containing human serum albumin microspheres was prepared by heat treatment and sonication of an aqueous solution of human serum albumin (1% w/v) in the presence of perfluoropropane gas, in accordance with the disclosure of WO-A-9501187.

Preparation 3—Lipid-Stabilised Sulphur Hexafluoride Microbubbles

A contrast agent formulation comprising lipid-stabilised sulphur hexafluoride microbubbles was prepared by addition of 0.9% saline to a lyophilisate of pharmaceutical grade polyethylene glycol 4000, distearoyl-phosphatidylcholine and dipalmitoylphosphatidylglycerol stored under an atmosphere of sulphur hexafluoride, in accordance with the disclosure of Schneider et al. in Invest. Radiol. 30(8) (1995), pp. 451-457.

Preparation 4—Lipid-Stabilised Perfluorobutane Microbubbles

The procedure of Preparation 3 was repeated except that a lyophilisate stored under an atmosphere of perfluorobutane was employed.

Preparation 5—Perfluoropropane-Containing Liposomes

Dipalmitoylphosphatidylcholine, dipalmitoylphosphatidylethanolamine coupled to polyethylene glycol 5000 (weight ratio 20:80) and dipalmitoylphosphatidic acid in a mole ratio of 82:8:10 were heated to 45EC in an aqueous carrier solution and sterile filtered (#0.22 μm filter), whereafter the solution was placed in a vial and allowed to cool to room temperature. The vial was subjected to vacuum to remove the gas content, pressurised with perfluoropropane, sealed and agitated on a shaker to generate perfluoropropane-containing liposomes.

Preparation 6—Perfluoropropane-Enhanced Sonicated Dextrose Albumin

The title contrast agent formulation was prepared by sonication of a mixture of aqueous human serum albumin (5% w/v) and aqueous dextrose (5% w/v) in the presence of perfluoropropane gas, in accordance with the disclosure of WO-A-9638180.

### EXAMPLE 1

### Open Chest Imaging Using Phosphatidylserine-Encapsulated Perfluorobutane Microbubbles and Adenosine

A midline sternotomy was performed on an anaesthetised 20 kg mongrel dog and the anterior pericardium was removed. A short axis view of the heart was imaged with an ATL HDI-3000 scanner, using a P5-3 transducer in harmonic mode; a 30 mm silicone rubber spacer having low ultrasound attenuation was placed between the transducer and the anterior surface of the heart. ECG gating was used so as to acquire one image in each end-systole. Ultrasound contrast agent from Preparation 1 (0.15 μl microbubbles/kg body weight) was then injected intravenously and allowed to equilibrate in the blood pool for 60 seconds, at which time stable enhancement of the whole imaged myocardium was observed, with moderate blood pool ultrasound attenuation. Adenosine (3 mg/ml in 0.9% saline; 150 μg/kg body weight) was then injected as an intravenous bolus; 7 seconds later a distinct general increase in echo intensity from the myocardium was observed, the effect lasting for some 15 seconds.

### EXAMPLE 2

### Closed Chest Imaging Using Phosphatidylserine-Encapsulated Perfluorobutane Microbubbles and Adenosine

The procedure of Example 1 was repeated with a closed chest dog, using a parasternal transducer position. The resulting ultrasound images were less homogeneous as regards signal intensities than the images obtained in Example 1, as a result of acoustic effects of the intact chest wall. However, despite these inhomogeneities, a general increase in echo intensities comparable to that observed in Example 1 was seen after injection of adenosine.

### EXAMPLE 3

### Partial-Coronary Occlusion: Open Chest Imaging Using Phosphatidylserine-Encapsulated Perfluorobutane Microbubbles and Adenosine

The procedure of Example 1 was repeated except that an occluding snare was placed around the major branch of the left anterior descending coronary artery. An ultrasound transit time flow meter was applied to the same artery and the snare was adjusted to give a stable reduction in blood flow to about 75% of the normal value. Contrast agent and adenosine injections were then administered as in Example 1. Upon arrival of the adenosine bolus in the heart, a slight decrease in contrast effect was observed in the tissue areas affected by the occlusion, whereas an increase was seen in all other areas of the myocardium.

### Example 4

### Method of Image Processing

In order to analyse ultrasound images obtained according to the procedure of Example 1, the images were first converted from video images into grey level digital images (640×480 pixels) using a frame grabber. A fixed central region (399×399 pixels) covering the image sector was selected for further processing. The thus-obtained images were decimated by averaging 3×3 pixels into new images (133×133 pixels) and were then median filtered using a sliding region (5×5 pixels).

A single image obtained just before onset of adenosine-induced vasodilatation was selected as a geometrical template to which all other images were automatically adjusted to maximum pixel correlation by an affine transformation (6 degrees of freedom). Only pixels within a region of interest in the template image encompassing the left ventricle and its myocardium were used for calculating maximum pairwise pixel grey level correlation.

6-10 baseline images from before the appearance of adenosine effects were averaged to give a representative baseline image, and a similar number of images at peak adenosine effect were likewise averaged. The two thus-obtained averaged images were then subtracted and the difference was colour coded. Since the grey levels of the digitised ultrasound images had a logarithmic dependency on signal intensity, the result of this subtraction is a dimensionless measure of relative changes in signal intensity. The colour coding was selected to represent the range −4 dB (blue colour) to +4 dB (red colour).

### EXAMPLE 5

### Processing of Images from Open Chest Investigation

Images were acquired as described in Example 1 and processed according to the method of Example 4. The normal myocardium was depicted with a homogeneous colour indicating a signal increase of some 3-4 dB.

### EXAMPLE 6

### Processing of Images from Closed Chest Investigation

Images were acquired as described in Example 2 and processed according to the method of Example 4. The normal myocardium was depicted with a homogeneous color indicating a signal increase of some 3-4 dB.

### EXAMPLE 7

### Processing of Images from Open Chest Investigation with Partial Coronary Occlusion

Images were acquired as described in Example 3 and processed according to the method of Example 4. The region of myocardial tissue affected-by the simulated coronary stenosis was in a colour indicating a 1-3 dB decrease in signal intensity following injection of adenosine, whilst the normal myocardium showed a corresponding 2-3 dB increase in signal intensity.

### EXAMPLE 8

### Imaginq with Perfluoropropane-Containing Human Serum Albumin Microspheres and Adenosine

a) Open chest imaging is performed as in Example 1 except that ultrasound contrast agent from Preparation 2 is employed.

b) Closed chest imaging is performed as in Example 2 except that ultrasound contrast agent from Preparation 2 is employed.

c) Open chest imaging with a partial coronary occlusion is performed as in Example 3 except that ultrasound contrast agent from Preparation 2 is employed.

### EXAMPLE 9

### Processing of Images Obtained Using Perfluoropropane-Containing Human Serum Albumin Microspheres and Adenosine

a-c) The method of Example 4 is used to process the images obtained according to Example 8(a)-(c).

### EXAMPLE 10

### Imaging with Lipid-Stabilised Sulphur Hexafluoride Microbubbles and Adenosine

a) Open chest imaging is performed as in Example 1 except that ultrasound contrast agent from Preparation 3 is employed.

b) Closed chest imaging is performed as in Example 2 except that ultrasound contrast agent from Preparation 3 is employed.

c) Open chest imaging with a partial coronary occlusion is performed as in Example 3 except that ultrasound contrast agent from Preparation 3 is employed.

### EXAMPLE 11

### Processing of Images Obtained Using Lipid-Stabilised Sulphur Hexafluoride Microbubbles and Adenosine

a-c) The method of Example 4 is used to process the images obtained according to Example 10(a)-(c).

### EXAMPLE 12

### Imaging with Lipid-Stabilised Perfluorobutane Microbubbles and Adenosine

a) Open chest imaging is performed as in Example 1 except that ultrasound contrast agent from Preparation 4 is employed.

b) Closed chest imaging is performed as in Example 2 except that ultrasound contrast agent from Preparation 4 is employed.

c) Open chest imaging with a partial coronary occlusion is performed as in Example 3 except that ultrasound contrast agent from Preparation 4 is employed.

### EXAMPLE 13

### Processing of Images Obtained Using Lipid-Stabilised Perfluorobutane Microbubbles and Adenosine

a-c) The method of Example 4 is used to process the images obtained according to Example 12(a)-(c).

### EXAMPLE 14

### Imaging with Perfluoropropane-Containing Liposomes and Adenosine

a) Open chest imaging is performed as in Example 1 except that ultrasound contrast agent from Preparation 5 is employed.

b) Closed chest imaging is performed as in Example 2 except that ultrasound contrast agent from Preparation 5 is employed.

c) Open chest imaging with a partial coronary occlusion is performed as in Example 3 except that ultrasound contrast agent from Preparation 5 is employed.

### EXAMPLE 15

### Processing of Images Obtained Using Perfluoropropane-Containing Liposomes and Adenosine

a-c) The method of Example 4 is used to process the images obtained according to Example 14(a)-(c).

### EXAMPLE 16

### Imaging with Perfluoropropane-Enhanced Sonicated Dextrose Albumin and Adenosine

a) Open chest imaging is performed as in Example 1 except that ultrasound contrast agent from Preparation 6 is employed.

b) Closed chest imaging is performed as in Example 2 except that ultrasound contrast agent from Preparation 6 is employed.

c) Open chest imaging with a partial coronary occlusion is performed as in Example 3 except that ultrasound contrast agent from Preparation 6 is employed.

### EXAMPLE 17

### Processing of Images Obtained Using Perfluoropropane-Enhanced Sonicated Dextrose Albumin and Adenosine

a-c) The method of Example 4 is used to process the images obtained according to Example 16(a)-(c).

### EXAMPLE 18

### Partial Coronary Occlusion: Open Chest Imaging Using Phosphatidylserine-Encapsulated Perfluorobutane Microbubbles and Dobutamine

The procedure of Example 3 is repeated except that the bolus injection of adenosine is replaced with a pumpcontrolled infusion of dobutamine at a rate of 15 μg/kg/min. One minute after the start of the infusion, an increase in contrast effect is observed in the myocardium, except in areas supplied by the stenotic artery.

### EXAMPLE 19

### Partial Coronary Occlusion: Open Chest Imaging Using Phosphatidylserine-Encapsulated Perfluorobutane Microbubbles and Arbutamine

The procedure of Example 3 is repeated except that the bolus injection of adenosine is replaced with a pumpcontrolled infusion of arbutamine at a rate of 0.4 μg/kg/min. One minute after the start of the infusion, an increase in contrast effect is observed in the myocardium, except in areas supplied by the stenotic artery.

### EXAMPLE 20

### Imaging in Man Using Phosphatidylserine-Encapsulated Perfluorobutane Microbubbles and Adenosine

A 45 year old male patient with an angiographically verified 80% left anterior descending arterial stenosis was given an intravenous injection of 1 ml of a perfluorobutane microbubble suspension prepared as in Preparation 1. The heart was imaged with an ATL HDI-5000 scanner and a P4-2 transducer, using ECG-gated (every second end-systole) pulse inversion imaging; the mechanical index was 0.6. The heart was imaged from an apical 2-chamber view. A stable contrast effect in all areas of the myocardium was obtained about one minute after injection of the contrast agent. Intravenous infusion of adenosine at a rate of 140 μg/kg/min was then started, and a sequence of 30 images, of which 10 were before the onset of adenosine effects in the heart, were stored in digital format. The images were processed as in Example 4, but without the initial video grabbing steps. The resulting colour image showed a 2-3 dB increase in signal intensity in normal regions of the myocardium, while myocardial regions supplied by the stenotic artery showed a 1-2 dB decrease in signal intensity. The procedure was repeated using a 4 chamber view, with similar results.

1. A method for detection of abnormalities in vasculated tissue within a human or non-human animal subject which comprises:
(A) injecting a substantially free-flowing contrast or tracer agent into the vascular system of said subject so as to generate a substantially steady state distribution of said agent in the blood stream of said subject during the steps of:
(i) generating one or more first images in respect of vasculated tissue in a target area;
(ii) inducing vasomodification within said target tissue; and
(iii) generating one or more second images in respect of said vasomodified target tissue, said one or more first images and said one or more second images being generated as parts of a single overall imaging sequence; and
(B) comparing said first and second images to identify any local variations in the change in signal intensity resulting from vascular volume changes induced by said vasomodification.

2. A method as claimed in claim 1 wherein the first and second images are compared by division or subtraction of signal intensity parameters.

3. A method as claimed in claim 1 wherein the images are generated by magnetic resonance imaging, X-ray imaging or a nuclear tracer technique.

4. A method as claimed in-claim 1 wherein the images are generated by ultrasound imaging.

5. A method as claimed in claim 4 wherein the contrast agent comprises microbubbles of gas stabilised by a coalescence-resistant surface membrane, a filmogenic protein, a polymer material, a non-polymeric and non-polymerisable wall-forming material or a surfactant.

6. A method as claimed in claim 5 wherein said surfactant comprises at least one phospholipid.

7. A method as claimed in claim 5 wherein at least 75% of said surfactant comprises a phosphatidylserine.

8. A method as claimed in claim 4 wherein the contrast agent comprises gas-containing microparticles.

9. A method as claimed in claim 8 wherein said microparticles comprise at least one carbohydrate.

10. A method as claimed in claim 5 wherein the gas comrises a perfluorocarbon or a sulfur fluoride.

11. A method as claimed in claim 10 wherein said gas comprises sulphur hexafluoride, perfluoropropane, perfluorobutane or perfluoropentane.

12. A method as claimed in claim 4 wherein perfusion-weighted images are generated using ultrasound irradiation at an intensity which causes destruction of the contrast agent.

13. A method as claimed in claim 1 wherein said vasomodification is induced by administration of one or more substances selected from vasodilators, vasoconstrictors, hormones, local signal substances and receptor blockers.

14. A method as claimed in claim 13 wherein vasomodification is induced by administration of an endogenous or metabolic vasodilator.

15. A method as claimed in claim 14 wherein said vasodilator is adenosine.

16. A method as claimed in claim 13 wherein said vasomodification is induced by administration of a beta receptor agonist.

17. A method as claimed in claim 16 wherein said beta receptor agonist is dobutamine or arbutamine.
